(19) United States
(12) Patent Application Publication
(10) Pub. No.: US 2010/0129413 A1
(43) Pub. Date: May 27, 2010

(54) METHOD OF TREATING AND PREVENTING VIRAL INFECTIONS

(75) Inventors: James Komorowski, Trumbull, CT (US); Barry Hurwitz, Miami, FL (US)

Correspondence Address: KATTEN MUCHIN ROSENMAN LLP, 575 MADISON AVENUE, NEW YORK, NY, 10022-2585, US

(73) Assignee: NUTRITION 21, INC., Purchase, NY (US)

(21) Appl. No.: 12/520,267

(22) PCT Filed: Dec. 19, 2007
(86) PCT No.: PCT/US07/88051

§ 371 Date: Jan. 20, 2010

Related U.S. Application Data
(60) Provisional application No. 60/877,699, filed on Dec. 28, 2006.
(60) Provisional application No. 60/880,550, filed on Jan. 16, 2007.

(51) Int. Cl. A61F 13/00; A61K 33/04; A61K 9/127; A61K 36/062; A61P 31/18

(52) U.S. Cl. 424/422; 424/702; 424/450; 424/93.51

(57) ABSTRACT

A method for treatment of a viral infection in a mammal comprising a step of administering a composition containing an amount of selenium (Se) effective to raise the serum level of the mammal to at least 15 μg/L above baseline.

### BACKGROUND

It has been shown previously that people who have been infected with the human immunodeficiency virus (HIV) typically have lower serum levels of selenium (Se). Though this fact has been well documented, the reasons behind it and its effect on the course of HIV in patients has not been extensively studied. Review of the current literature provides contradictory or inconclusive rationale for this reduced level of Se. Among the competing theories is that the reduced levels of Se increase the susceptibility of viral infections. Another theory is that viruses may in fact use the Se for replication and thus cause the reduction in serum levels. Due to the inconclusive nature of the current literature regarding the cause/effect of reduced Se levels, there was a need to study the virus Se relationship.

### DETAILED DESCRIPTION

Based on the forgoing need a study was conducted of people infected with HIV. This study dosed the participants with amounts of Se and various serum levels were achieved. The study involved some 310 randomized participants of which 141 were treated with Se and 121 were given a placebo. Based on analysis it was determined that those patients who through dosing achieved an increase in serum selenium level of at least 15 μg/l experienced a significant difference in viral load change as compared to placebo group and non-responding patients. Those patients who through dosing achieved an increase in serum selenium level of at least 26.1 μg/l experienced a significant suppression of viral burden and an increase in CD4 count. Those patients who when dosed experienced an increase in serum selenium level of below the 15 μg/l did not appear to show a significant suppression of viral burden, but experienced some suppression of viral burden depending on the serum level achieved. As this is a serum level, it can be achieved through any treatment method such as orally, parentally, intraperitoneally, intrathecally, intraarterially, sublingually, transbuccally, intraoccularly, intranasally, rectally, intravaginally, transdermally, liposomally, via inhalation, locally via catheter or stent. Though not specifically a feature of this study it will be understood by those of skill in the art that such treatment, where viral burden is suppressed, will likely reduce total viral count and may provide preventative effects to HIV.

As part of the study the Se was provided to the subjects by administering 200 μg of selenium as Selenomax high selenium yeast made and sold by Nutrition 21, Inc. This product is made to contain 1200 μg Se/g yeast.

Though the imunno-system benefits of Se were known, heretofore it was not known that increased serum levels of Se could suppress viral burden. Finally, though reduced levels of Se in HIV infected people had been noted, it was not known that by increasing the level of Se in serum to 26.1 μg/l and above could significantly suppress the viral burden.

The study attached herewith which substantiates these claims, “Suppression of Human Immunodeficiency Virus Type 1 Viral Load With Selenium Supplementation,” which is incorporated herein by reference, and appeared in the Jan. 22, 2007 issue of Archives of Internal Medicine.

1. A method for treatment of a viral infection in a mammal comprising a step of administering a composition containing an amount of selenium (Se) effective to raise the serum level of the mammal to at least 15 μg/L above baseline.

2. The method of claim 1, wherein the Se is administered orally, parentally, intraperitoneally, intrathecally, intraarterially, sublingually, transbuccally, intraoccularly, intranasally, rectally, intravaginally, transdermally, liposomally, via inhalation, locally via catheter or stent.

3. The method of claim 1, wherein the Se is a bioavailable form.

4. The method of claim 1, wherein the Se is a high selenium yeast.

5. The method of claim 1, wherein the Se is L-selenomethiomne.

6. The method of claim 1, wherein the Se is a high selenium yeast containing approximately 200 μg of selenium.

7. A method of stabilizing the replication of a viral infection including HIV, wherein a mammal in need thereof is administered a composition comprising selenium to achieve an increase in serum selenium level of at least 15 μg/L or more.

8. A method of treating a viral infection including HIV, wherein a mammal in need thereof is administered a composition comprising selenium to achieve an increase in serum selenium level of at least 15 μg/L or more.

9. A method of preventing a viral infection including HIV, wherein a mammal in need thereof is administered a composition comprising selenium to achieve an increase in serum selenium level of at least 15 μg/L or more.

10. A method for treatment of a viral infection in a mammal comprising a step of administering a composition containing an amount of selenium (Se) to raise the serum level of the mammal to an effective level that reduces the viral burden.

11. A method of stabilizing the replication of a viral infection including HIV, wherein a mammal in need thereof is administered a composition comprising selenium (Se) to achieve a serum level effective to suppresses the viral burden.

12. A method of treating a viral infection including HIV, wherein a mammal in need thereof is administered a composition comprising selenium (Se) to achieve a serum level effective to reduces the viral burden.

13. A method of achieving a serum selenium level effective to prevent a viral infection including HIV, wherein a mammal in need thereof is administered a composition comprising selenium (Se).

14. The method of claim 2, wherein the oral dosage form is a tablet, capsule, lozenge, or troche.

15. A method of raising CD4 count in an individual comprising the steps of administering a composition containing an amount of selenium (Se) effective to raise the serum level of the mammal to achieve an increase in CD4 count.
